(19) United States
(12) Patent Application Publication
(10) Pub. No.: US 2021/0030911 A1
(43) Pub. Date: Feb. 4, 2021

(54) VISUAL AND OLFACTORY PRESENTATION DEVICE

(71) Applicant: Munirhusein Adat, Plainsboro, NJ (US)

(72) Inventor: Munirhusein Adat, Plainsboro, NJ (US)

(21) Appl. No.: 16/947,350

(22) Filed: Jul. 29, 2020

Related U.S. Application Data
(60) Provisional application No. 62/922,266, filed on Aug. 1, 2019.

(51) Int. Cl. A61L 9/03; G09F 1/02; A61L 9/12

(52) U.S. Cl. CPC A61L 9/03; G09F 1/04; A61L 9/12; G09F 1/02

(57) ABSTRACT

A visual and olfactory presentation device combining a three-dimensional display card with one or more scented container attached thereto is provided. The visual and olfactory presentation device enables the synergetic effect of a visual presentation from an indicia area along the display card and an olfactory presentation from a scent formulation housed in each scented container. The scented container(s) may be disposed along the surface of the display card in such a way to enable a kickstand configuration along a supporting surface so that the indicia area is upwardly facing and enveloped in the scented formulation.

### CROSS-REFERENCE TO RELATED APPLICATION

This application claims the benefit of priority of U.S. provisional application No. 62/922,266, filed 1 Aug. 2019, the contents of which are herein incorporated by reference.

### BACKGROUND OF THE INVENTION

The present invention relates to visual and olfactory presentation devices and, more particularly, a three-dimensional display card with a scented container attached thereto, enabling the synergetic effect of visual and olfactory presentation for leaving a lasting impression as well as a convenient and low-cost sampling and diffusion of perfumed products.

Scented cards or even business cards are normally looked at once and then thrown away or stored in a forgettable file. Obviously, this frustrates the purpose of the free offering—creating a lasting impression—and as a result wastes time, money, and opportunities for making meaningful impressions on the recipient.

Liquid perfumes and formulations are costly to sample and/or difficult to present to the customer—e.g., perfume samples in stores can only be used (and thus evaluated) once and in an artificial environment. Air-fresheners such as the cardboard tree shaped gels only contains one scent, they do not last long, and/or do not offer the customer any flexibility of choice.

As can be seen, there is a need for a visual and olfactory presentation device combining a three-dimensional display card with a scented container attached thereto, enabling the synergetic effect of visual and olfactory presentation for leaving a lasting impression. The present invention would enable both perfumes to be sampled and used a multitude of times and, when combined with a display card, enhance the memorability of information conveyed by way of the display surface. The visual and olfactory presentation device embodied by the present invention has a display surface portion operatively associated with one or more scent container portions in such a way that the latter props up the former.

The present invention allows much more flexibility in using multiple scents and associating these scents with names, brands and/or other information in a much cost-effective way. As a result, the present invention enables air-fresheners users flexibility in usage of their scents, while decreasing the chances of the scent(s) being thrown away as the container with the scented formula also acts as a stand so the card can be propped on a supporting surface in a presentable manner while being readily available to use.

The display surface portion can have more than one container portion attached so the user can have the flexibility of being able to use one or more scents. For perfume makers and suppliers this type of device can also be used to sample one or more perfume scent as the display portion provide “free” space for graphics or information to enhance the memorability of the product. The presenter does not have to be there as the card can contain all the necessary information.

### SUMMARY OF THE INVENTION

In one aspect of the present invention, a visual and olfactory presentation (VOP) device including the following: a display card having a planar configuration with a card thickness at least one tenth a length or a width; an indicia area on one surface of the planar configuration; and one or more scented containers operatively associated with the display card in an off-center manner so that the display card can be positioned in a kickstand configuration with the indicia area facing upwardly.

In another aspect of the present invention, a method for creating a visual and olfactory presentation, the method includes the following: providing the above-mentioned VOP device; and propping the indicia area against a supporting surface by way of the kickstand configuration.

These and other features, aspects and advantages of the present invention will become better understood with reference to the following drawings, description and claims.

### BRIEF DESCRIPTION OF THE DRAWINGS

FIG. 1 is a rear perspective view of an exemplary embodiment of the present invention;

FIG. 2 is a rear perspective view of an exemplary embodiment of the present invention;

FIG. 3 is an exploded perspective view of an exemplary embodiment of the present invention;

FIG. 4 is a section view of an exemplary embodiment of the present invention, taken along line 4-4 in FIG. 1;

FIG. 5 is a rear perspective view of an exemplary embodiment of the present invention, illustrating the use of container 10 and lid 18 as a display stand;

FIG. 6 is a side view of an exemplary embodiment of the present invention in a display position;

FIG. 7 is a perspective view of an exemplary embodiment of the present invention;

FIG. 8 is a section view of an exemplary embodiment of the present invention, taken along line 8-8 in FIG. 7;

FIG. 9 is a rear perspective view of an exemplary embodiment of the present invention;

FIG. 10 is a rear perspective view of an exemplary embodiment of the present invention;

FIG. 11 is a section view of an exemplary embodiment of the present invention, taken along line 11-11 in FIG. 9;

FIG. 12 is a side view of an exemplary embodiment of the present invention in a display position utilizing a ‘kickstand’ configuration;

FIG. 13 is a perspective view of an exemplary embodiment of the present invention, shown laid flat with one lid 44 removed; and

FIG. 14 is a perspective view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a visual and olfactory presentation device combining a three-dimensional display card with one or more scented container attached thereto, enabling the synergetic effect of a visual presentation from an indicia area along the display card and an olfactory presentation from a scent formulation housed in each scented container. The scented container(s) may be disposed along the surface of the display card in such a way to enable a kickstand configuration along a supporting surface so that the indicia area is upwardly facing and enveloped in the scented formulation.

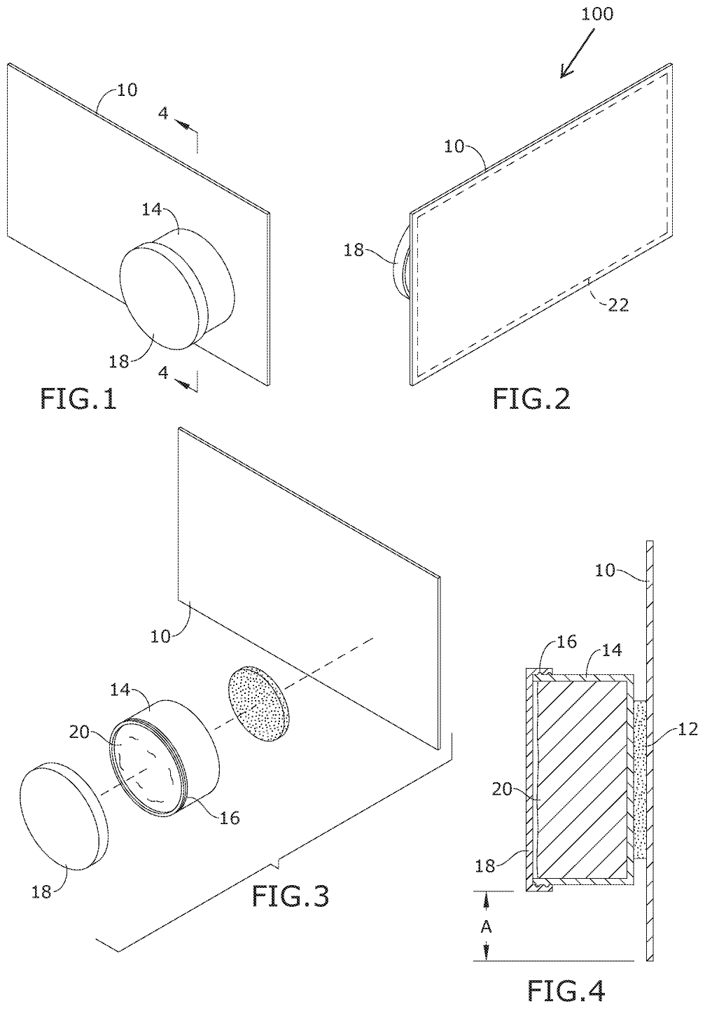

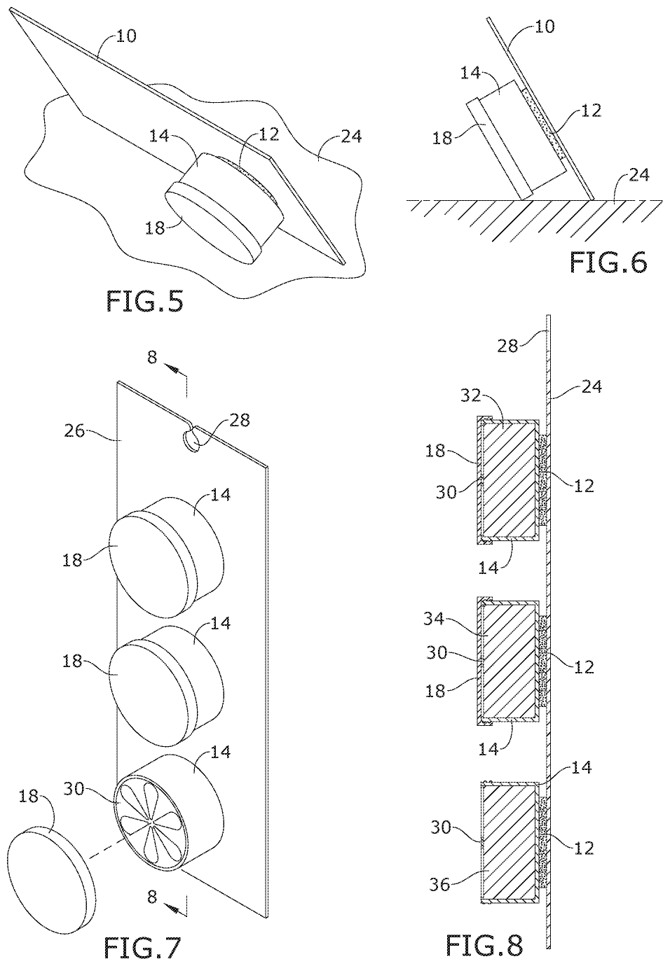

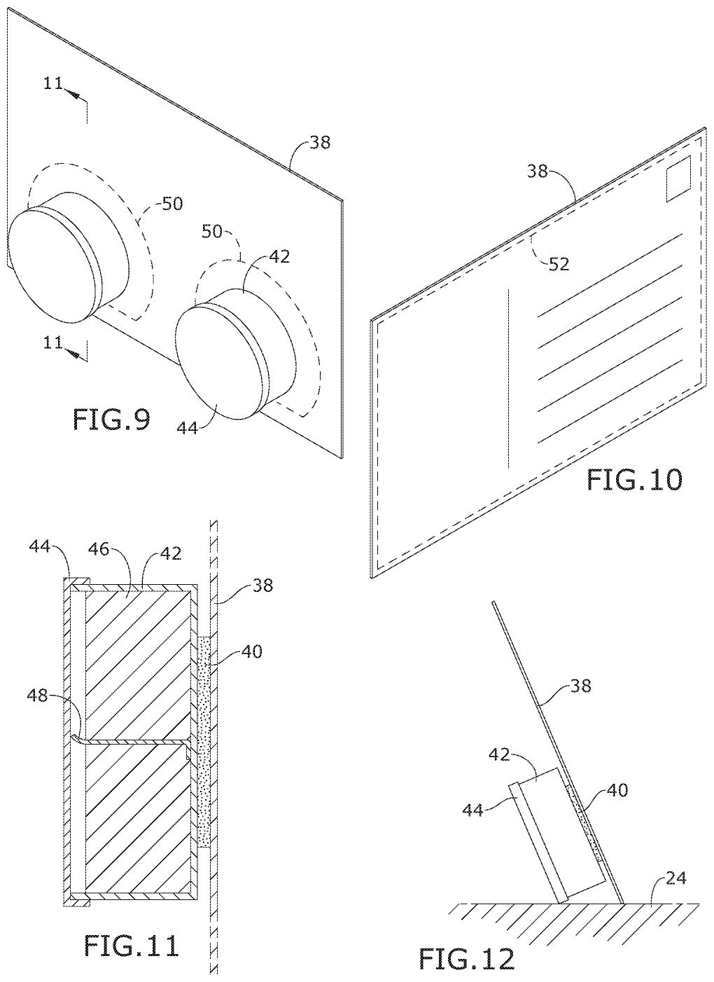

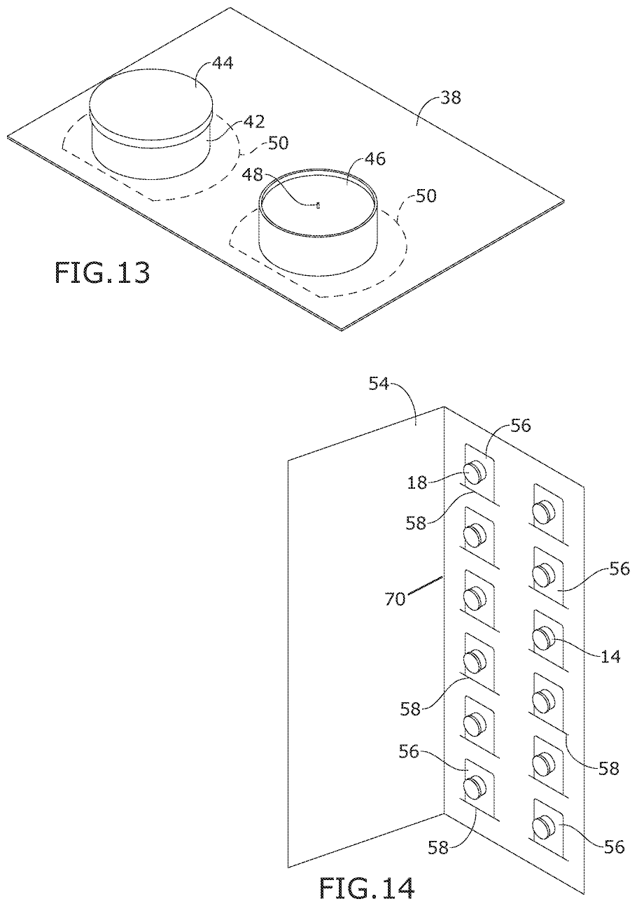

Referring now to FIGS. 1 through 14, the present invention may include a visual and olfactory presentation device 100 (or VOP device 100). The VOP device 100 may include a display card 10, 26, 38, 56, or 54 coupled to one or more scent containers 14 and/or 42. Each scent container 14 or 42 may be connected to the display card 10, 26, 38, 56, or 54 by way of an adhesive 12 or 40, such as double-sided tape, glue or the like. The scent container 14 or 42 may house a scented formulation 20, 32, 34, 36 or a combination thereof in a compartment. The scented formulation 20, 32, 34, 36 may include skin creams, lip balms, solid perfume or the like. The scented formulation 34 may include scent grills 30. The scented formulation may be in candle form 46 with a wick 48. In certain embodiments, a lid 18 or 44 may close off access to the compartment. Threading 16 may be the structure that facilitates the lid 18 or 44 securely attaching to the scent container 14 or 42 when closing off the access to the compartment.

The display card 10, 26, 38, 56, or 54 may be, generally but not necessarily, elongated having a length at least twice its width. The 10, 26, 38, 56, or 54 may be, generally but not necessarily, planar having a thickness one-tenth or less its width. The display card 10, 26, 38, 56, or 54 may provide a hanger slot 28 or a fold 70 in some embodiments. Each display card 10, 26, 38, 56, or 54 may provide an indicia area 22, 50, or 52 for providing messages or information, including branding, slogans, instructions, inspirational messages, artistic designs and the like. In some embodiments, the display card 38 may take the traditional form of a U.S. postal card.

The scent container 14 or 42 may be disposed off-center and/or adjacent to a corner and/or a periphery of the display card 10, 26, 38, 56, or 54 in such a way that the latter can be propped up on a supporting surface 24 wherein the former acts as a ‘kickstand’, as illustrated in FIG. 6. In certain embodiments, the scent container(s) 14 and/or 42 may be spaced between 0-0.3 inches apart of the periphery of the display card 10, 26, 38, 56, or 54 in order for the whole display card 10, 26, 38, 56, or 54 to be propped up. Also, the scented container 14 or 42 may have thickness at least four time that of the display card 10, 26, 38, 56, or 54 for facilitating the kickstand configuration. The indicia area 22 or 52 in certain embodiments being disposed on a surface of the display card 10 or 38, respectively, opposite the scent container 14 or 42 so that the scent container 14 or 42 does not juxtapose with or obscure the indicia area 22 or 52.

In certain embodiments, the display card 70 may have a plurality of spaced apart slots 58, wherein each slot 58 is dimensioned and adapted to receive a singular display card 56, as illustrated in FIG. 14.

A method of using the present invention may include the following. The VOP device 100 disclosed above may be provided. For fragrance or product sampling one can either distribute single display cards with the identifier of the product or one could employ a card with multiple scent containers that can then be distributed to illustrate the range of scents or products. For display cards with candle scent formulations, the user would lay the display card on a flat surface and light the wick, wherein the indicia area is on the same surface as the scented container. For solid scent formulations, one could undo the lid of the scent container and use their fingertips to dab some sample on to skin. For air-freshener cards embodiments, one would unscrew the lid to reveal a scent formulation with a plastic insert grill across the opening. The display card could hold one or more perfumed containers that could be opened according to the user's needs.

Additionally, the present invention could be used for unique scented greeting cards where the display card would contain a scent depending on the occasion. For example, a rose scent for Valentine's day. Alternatively, the VOP device 100 could also be used by the pharmaceutical industry as a cost-effective way to sample medicines. The VOP device 100 could also be used in the essential oil/aromatherapy industry to give people a low cost and effective way for selfcare by filing the container with solid formula containing essential oils or blends thereof.

It should be understood, of course, that the foregoing relates to exemplary embodiments of the invention and that modifications may be made without departing from the spirit and scope of the invention as set forth in the following claims.

What is claimed is:

1. A visual and olfactory presentation (VOP) device, comprising:
a display card having a planar configuration with a card thickness at least one tenth a length or a width;
an indicia area on one surface of the planar configuration; and
one or more scented containers operatively associated with the display card in an off-center manner so that the display card can be positioned in a kickstand configuration with the indicia area facing upwardly.

2. The VOP device of claim 1, wherein each scented container is within 0.3 inches of less from a periphery of the display card.

3. The VOP device of claim 2, wherein each scented container has a container thickness at least four times the card thickness.

4. The VOP device of claim 1, wherein each scented container contains a scented formulation.

5. The VOP device of claim 4, wherein the scented formulation is in a candle form.

6. The VOP device of claim 4, wherein the indicia area circumscribes the scented container.

7. The VOP device of claim 4, wherein the scented formulation includes air-freshener grilling.

8. The VOP device of claim 1, wherein the display card provides a plurality of slots; wherein a second display card is dimensioned to slidably engage each of the plurality of slots, and wherein each scented container is connected to the second display card.

9. A method for creating a visual and olfactory presentation, the method comprising:
providing the VOP device of claim 1; and
propping the indicia area against a supporting surface by way of the kickstand configuration.
